(19) United States
(12) Patent Application Publication
(10) Pub. No.: US 2018/0311123 A1
(43) Pub. Date: Nov. 1, 2018

(54) COSMETIC COMPOSITION HAVING IMPROVED SMEARING PROPERTY

(71) Applicant: AMOREPACIFIC CORPORATION, Seoul (KR)

(72) Inventors: Choon Bok JEONG, Yongin-si, Gyeonggi-do (KR); Yu Jin KANG, Yongin-si Gyeonggi-do (KR); Kyung Nam KIM, Yongin-si Gyeonggi-do (KR); Yeong Jin CHOI, Yongin-si Gyeonggi-do (KR)

(73) Assignee: AMOREPACIFIC CORPORATION, Seoul (KR)

(21) Appl. No.: 15/756,840

(22) PCT Filed: Sep. 13, 2016
(86) PCT No.: PCT/KR2016/010340

§ 371 Date: Mar. 1, 2018

(30) Foreign Application Priority Data
Sep. 18, 2015 (KR) 10-2015-0132524

(51) Int. Cl. A61K 8/06; A61K 8/25; A61K 8/73; A61K 8/891; A61Q 1/12; A61Q 17/04

(52) U.S. Cl. CPC A61K 8/064; A61K 8/25; A61K 8/731; A61K 8/732; A61K 2800/54; A61Q 1/12; A61Q 17/04; A61K 2800/48; A61K 8/891

(57) ABSTRACT

The present invention relates to cosmetic composition having an improved smearing property. The cosmetic composition according to the present invention is directed to a water-in-oil type cosmetic composition containing a film former in an external phase, the composition being characterized by further containing a thickener in an internal phase. The thickener serves to form a kind of a bridge between the skin and the film former in an external phase, of which the affinity with the skin may deteriorate. According to the present invention, the smearing of the cosmetic composition can be improved more pragmatically and effectively by a combination with an external-phase film application manner of the prior art.

### TECHNICAL FIELD

This application claims priority to and the benefit of Korean Patent Application No. 10-2015-0132524, filed on Sep. 18, 2015, the entire contents of which are incorporated herein by reference.

The present invention relates to a cosmetic composition containing a film former in an external phase, characterized in that a thickener is further included in an internal phase. The cosmetic composition of the present invention can be applied to make-up cosmetic products and other similar fields since the transfer is small after application to the skin.

### BACKGROUND ART

Make-up cosmetic compositions are used routinely and should have their suitable functions without causing discomfort to life. However, in the case of most cosmetic compositions that are applied to the skin in the form of over applying, when there is an external contact to the skin, cosmetics are often smeared to the contact or removed due to its characteristics, and on the other hand, the migration of cosmetics on the skin is also caused. For example, it may cause discomfort by leaving dirt or stain on the clothes or the liquid crystal of the mobile phone, and particularly, in the case of water-in-oil (W/O type) cosmetics having color, such as pact or foundation, it is very difficult to simply clean using water. Additionally, due to migration, agglomeration, erasure, etc. of the cosmetic composition applied on the skin, there is always an onerousness of correcting the makeup or re-applying the cosmetics.

To prevent this inconvenience, a series of attempts have been made in the past to improve the transfer of cosmetics, especially water-in-oil cosmetic compositions. However, these attempts have focused mainly on the use of a film former in the external phase of the cosmetic composition and have been studied in the direction of directly or indirectly increasing or supplementing its content, and thus were substantially merely to add some variation to specific content, combination, location and the like of the film former.

For example, in order to improve the feeling of use at the time of film formation after the evaporation of oil, certain copolymers and monomers were used as a film former, or in the case of a slightly improved form, forms containing the film former even in the internal phase have also been proposed. However, all of them did not show a satisfactory level of transfer improvement effect and also have caused a problem of causing excessive skin dryness. In addition, considering the compatibility with other constituents of the composition, the use of the product, the feeling of use, etc., increasing the amount of film former infinitely also has its limitations, and accordingly, it was very difficult to improve the transfer.

In order to solve the above-described conventional problems, the inventors of the present invention have made considerable efforts and studies to prepare a new type of cosmetic composition capable of preventing the transfer, and as a result, have found that for the conventional water-in-oil cosmetic composition with the film former applied to the external phase, when a thickener is further combined with its internal phase, a transfer improvement effect occurs.

Regarding the use of the thickener in the internal phase, it has been common in the past to use the thickener in an oil-in-water type (0/W type) emulsion composition, and in the case of a water-in-oil (W/O type) emulsion composition, even although the thickener was contained in the internal phase, it has never been used for transfer improvement purpose along with the film former in the external phase, and thus the inventors of the present invention have come up with a new solution by completing the present invention while moving away from the conventional solution for the transfer improvement of cosmetic composition.

### PRIOR ART LITERATURE

### Patent Literature

Korea Patent Publication No. 10-0797072, COSMETIC COMPOSITIONS FOR IMPROVING THE PERFORMANCE OF TRANSFER-RESISTANT AND LONG-WEARING

### DISCLOSURE

### Technical Problem

The present invention has been made in order to solve the problems of the conventional cosmetic composition.

First, it is an object of the present invention to improve the transfer phenomenon of the conventional cosmetic composition. Particularly, it is a specific object of the present invention to overcome the limitations of the conventional method of controlling the transfer by using a film former in an external phase or an internal phase and to accomplish this through new means.

Second, it is another object of the present invention to stably apply the new means to a cosmetic composition.

### Technical Solution

In order to accomplish the above objects, the present invention provides a water-in-oil cosmetic composition containing a film former in an external phase characterized in that a thickener is further contained in an internal phase.

The type of the thickener contained in the internal phase of the cosmetic composition of the present invention is not particularly limited, but may be typically any one or more of saccharides, anionic thickener, and inorganic thickener. Preferably, the thickener may be one or a mixture of two or more selected from the group consisting of xanthan gum, carbomer, hydroxypropyl starch phosphate, sodium magnesium silicate, hydroxyethyl cellulose, hydroxyethyl acrylate/sodium acryloyldimethyl taurate copolymer. More preferably, the thickener may be sodium magnesium silicate and/or hydroxyethyl cellulose.

The thickener contained in the internal phase plays a role in forming a bridge between the film former in the external phase, which may have poor affinity with the skin, and the skin. Through this role, the affinity between the film former and the skin is increased and the transfer is effectively improved.

Meanwhile, the type and content of the film former contained in the external phase of the cosmetic composition of the present invention may follow without limitation any conventional or new methods.

The present invention provides a water-in-oil type cosmetic composition containing a film former in an external phase characterized in that a thickener is further contained in an internal phase.

At this time, the thickener contained in the internal phase may be at least one thickener selected from saccharides, anionic thickener and inorganic thickener.

At this time, the thickener may be at least one selected from the group consisting of xanthan gum, carbomer, hydroxypropyl starch phosphate, sodium magnesium silicate, hydroxyethyl cellulose, and hydroxyethyl acrylate/sodium acryloyldimethyl taurate copolymer.

At this time, the thickener may be at least one selected from the group consisting of sodium magnesium silicate and hydroxyethyl cellulose.

At this time, the content of the thickener may be 0.1 to 0.5% by weight based on the total weight of the composition.

At this time, the film former contained in the external phase may be trimethylsiloxysilicate or polypropylsilsesquioxane.

At this time, the water-in-oil cosmetic composition may have a transfer preventing effect.

At this time, the water-in-oil cosmetic composition may be at least one selected from the group consisting of foundation, pact, powder, makeup base, sunscreen, primer and concealer.

### Advantageous Effects

First, the present invention provides a cosmetic composition with improved transfer using the means of solving the above-mentioned problems.

Second, the present invention provides a cosmetic composition in which the stability of the composition is maintained while applying the means of solving the above-mentioned problems.

The present invention can overcome the conventional limitations pragmatically and simply by proposing a new method that can be used in combination with the conventionally used method, which can exhibit an improved effect while deviating from the conventionally used method.

### DESCRIPTION OF DRAWINGS

FIG. 1 is a graph showing the transfer improvement rates (%) of cosmetic compositions according to the present invention in comparison with the prior art. (A): Xanthan gum, (B): Carbomer, (C): Hydroxypropyl starch phosphate, (D): Sodium magnesium silicate, (E): Hydroxyethyl cellulose, (F): Hydroxyethyl acrylate/sodium acryloyldimethyl taurate copolymer.

### BEST MODE

Hereinafter, detailed descriptions for carrying out the present invention will be described. However, it is to be understood that the following description merely illustrates the most representative embodiments in order to facilitate understanding of the present invention, the scope of rights of the present invention is not limited thereto, and the present invention encompasses all ranges equivalent to those described below.

As used herein, ‘transfer’ means that apart from the stability of the formulation itself, when external physical contact is applied, such as when another object comes into contact with the skin, the cosmetic composition is smeared on the object or the application state of the cosmetic composition to the skin is lowered. This concept is clearly distinguished from ‘thixotropy’ in which the stability of the formulation itself changes and the application state is lowered over time due to sebum or sweat secreted from the skin itself, or environmental stimulation such as temperature or humidity. That is, transfer and thixotropy are properties that depend on different factors. In fact, there are many cosmetic compositions having good thixotropy but large transfer properties or having poor thixotropy but little transfer properties.

The present invention provides a water-in-oil cosmetic composition containing a film former in an external phase characterized in that a water-soluble thickener is further contained in an internal phase.

The cosmetic composition of the present invention has an effect of improving the transfer in comparison with the conventional type in which only the film former is applied to the external phase.

The thickener used in the present invention refers to a substance used for increasing the viscosity of the composition, for improving the physical stability of the emulsion formulation, or for improving the applicability by imparting rigidity, smoothness, soft touch or the like to the product when the product is applied to the human body.

The thickener can be largely divided into a water-insoluble thickener (lipid-soluble thickener) and a water-soluble thickener. The thickener used in the present invention can be a water-soluble thickener which can be dispersed in the water phase which is an internal phase. The water-soluble thickener can be divided into an inorganic thickener composed of metal oxide and the like and an organic thickener composed of the other water-soluble polymer material. Table 1 below shows representative water-soluble thickeners classified according to the above criteria.

TABLE 1

| | | | |
|---|---|---|---|
| Organic | Natural | Vegetable-based | Guar gum, Locust bean gum, Queen's seed, |
| thickeners | polymers | (Polysaccharides- | Carrageenan, Galactan, Gum arabic, |
| | | based) | Tragacanth gum, Pectin, Manna, Starch |
| | | Microorganism- | Xanthan gum, Dextran, Saxinoglucan, |
| | | based | Cadran, Hyaluronic acid |
| | | (Polysaccharides- | |
| | | based) | |
| | | Animal-based | Gelatin, Casein, Albumin, Collagen |
| | | (Proteins-based) | |
| | Semi- | Cellulose-based | Methyl cellulose, Ethyl cellulose, Hydroxy |
| | synthetic | | ethyl cellulose, Hydroxy propyl cellulose, |
| | polymers | | Carboxy methyl cellulose, Methyl hydroxy |
| | | | propyl cellulose |
| | | Starch-based | Soluble starch, Carboxy methyl starch, |
| | | | Methyl starch |
| | | Alginic acid-based | Alginic acid propylene glycol ester, Alginate |
| | | Others | Other polysaccharide derivatives |
| | Synthetic | Vinyl-based | Poly vinyl alcohol, Polyvinyl pyrrolidone, Poly |
| | polymers | | vinyl methyl ether, Carboxy vinyl polymer |
| | | | (carbomer), Sodium polyacrylate |
| | | Others | Poly ethylene oxide, Ethylene oxide, |
| | | | propylene oxide, Propylene oxide bromine |
| | | | copolymer |

| | | |
|---|---|---|
| Inorganic | Metal oxides | Bentonite, Laponite, Fine powder silicon |
| thickeners | | oxide, Colloidal alumina, Sodium magnesium |
| | | silicate |

The thickener of the present invention may include without limitation not only the thickener listed in table 1 but also any conventional thickener which can be included in the internal phase.

The thickener used in the present invention may be used in the form of one kind of thickener or as a mixture of two or more kinds. The thickener is preferably saccharides, anionic thickener, and inorganic thickener, and may be one or a mixture of two or more selected from the group consisting of xanthan gum, carbomer, hydroxypropyl starch phosphate, sodium magnesium silicate, hydroxyethyl cellulose (HEC) and hydroxyethyl acrylate/sodium acryloyldimethyl taurate copolymer, and more preferably may be sodium magnesium silicate and/or hydroxyethyl cellulose.

The content of the thickener used in the present invention is not necessarily limited to a specific range, and preferably may be 0.1 to 0.5% by weight based on the total weight of the composition.

Since each thickener differs in its usage and stability from one type to another, the thickener with the most suitable characteristics is selected and used in consideration of the specific purpose, type, use and effect of the cosmetic composition to be provided.

For example, the xanthan gum is a microorganism-derived thickener, and it is preferable that when used the xanthan gum is allowed to stand at −40° C. or less and gradually dissolve. The xanthan gum exhibits little change in viscosity depending on pH and remains stable, and exhibits a very uniform increasing effect in viscosity. However, since the xanthan gum is shiny and has a slippery feel, if the content of the xanthan gum is excessive, it is important, when used, to maintain proper content as it may cause migration on the skin and excessive gloss and lead to tugging. In addition, in the case of using alone, the xanthan gum may cause the phenomenon of white turbidity to deteriorate the visual effect, and thus considering this, the xanthan gum can be used with other thickener.

In addition, the carbomer is a synthetic polymer having a carboxy group mainly obtained by polymerizing acrylic acid, as an acidic polymer compound. The carbomer is one of the most widely used thickener because it has little variation in quality, has little viscosity change depending on lapse of time and temperature, is not significantly contaminated by microorganisms and has good viscosity increasing effect and good feeling of use. Specifically, the carbomers are divided into various types such as carbomer 400, carbomer 940, carbomer pregel and the like, and are somewhat different in their characteristics depending on the detailed types. For example, carbomer 400 is more soluble than carbomer 940 and stable in a wide pH range, has good feeling of use, and is safe against microorganisms and non-toxic. In addition, the carbomer pregel is easy to use and is specifically characterized by its ability to be used at the temperature of −60° C. or less without heating.

In addition, the hydroxyethyl cellulose is a water-soluble polymer obtained by making cellulose into alkaline cellulose using caustic soda and then neutralizing it by reaction with ethylene oxide, and purifying, drying and crushing it, and its viscosity depends on the degree of polymerization of the cellulose body. When added to water to increase viscosity, if the addition concentration is doubled, the viscosity usually becomes 5-10 times. In addition, the larger the molecular weight and the higher the concentration, the greater the structural viscosity. The aqueous solution is stable in the pH range of 2 to 12, and is particularly excellent in long-term storage stability in the pH range of 4 to 11. However, under strongly acidic or strongly alkaline conditions, hydrolysis and oxidative decomposition occur and the viscosity decreases. The hydroxyethyl cellulose has excellent compatibility with almost all inorganic salts and conventional water-soluble polymers and may be used in combination with them. It has excellent temperature stability, chemical resistance and heat resistance, and has no toxicity or irritation to human body.

On the other hand, the film former contained in the external phase in the constitution of the cosmetic composition according to the present invention is a kind of oil-soluble (also called liposoluble) polymer. They are commonly used with volatile oils in many cases. As the volatile oil evaporates, an adhesive film is formed on the skin from the remaining film former, and the film prevents cosmetic composition from transferring to the material that come into contact with the skin.

The specific type and application form of the film former may follow without limitation any conventional or new methods, and for example, any one or a mixture of two or more selected from the group consisting of acrylamide and polyquaternium, and modified polyvinyl pyrrolidone-based, aromatic hydrocarbon based, terpene based, polyisoprene based, polysaccharide based and acrylic acid based polymers and their copolymers may be used, and more specifically, trimethylsiloxy silicate, which is a kind of silicone resin, and polypropylsilsesquioxane may be used.

The total content of the film former is preferably 5 to 10% based on the total weight of the composition. When used in the form of a mixture of two or more, an appropriate combination ratio can be selected in consideration of the compatibility and effect between the materials. However, if the film former is used too little, the transfer improvement effect is too low. If the film former is used excessively, it should be noted that the film is too high and thick and thus burdens the skin.

In the past, the thickener contained in the internal phase has been commonly used for the oil-in-water type composition, but tended to be used relatively infrequently in the water-in-oil composition. In particular, if the thickener is contained in the internal phase of the water-in-oil composition, the thickener can be self-gelling depending on time, temperature and/or physical changes, and thus there was a problem of stabilization, and when used the product, the thickener required careful application since it can cause migration on the skin.

However, a certain type of thickener contained in the internal phase according to the present invention improves the transfer by complementing the affinity between the film former in the external phase and the skin, without showing the stability problem.

Describing in detail as for the transfer improvement effect, it is known that the film former is usually contained in the oil phase along with volatile oil and forms a film when the volatile oil evaporates after application to the skin, and thus prevents the transfer of the cosmetic composition. However, in the past, this film former has less affinity with the skin in many cases, resulting in a poor transfer improvement effect, and as a result, even when the content of the film former is intended to be increased, there is a problem of causing skin dryness and so on, and thus application of the film former is limited. Therefore, in the present invention, a thickener is contained in the internal phase, as a means for improving the affinity between the film former and the skin, and as a result, a kind of bridge is formed between the film former of the external phase and the skin. That is, the transfer is more effectively improved by adding the network formed by the thickener in the internal phase to the network which was originally formed between the film former in the external phase and the skin.

The cosmetic composition according to the present invention is not particularly limited in its formulation and can be used as any formulation as long as it is a water-in-oil cosmetic composition that requires the prevention of transfer. For example, the cosmetic composition can be prepared as lotion, milk lotion, moisture lotion, nutrient lotion, massage cream, nutrient cream, moisture cream, hand cream, foundation, make-up base, primer, essence, nutrient essence, pack, soap, cleansing foam, cleansing lotion, cleansing cream, body lotion, body cleanser and the like, and preferably it is more effective if it is formulated as formulations such as foundation, pact, makeup base, sunscreen, primer and concealer.

Since the cosmetic composition with improved transfer according to the present invention is applied to the skin and maintained in an evenly applied state even after external contact to the skin, a part of it is not clumped or erased on the skin, so it does not need to be reapplied frequently, and particularly, it is more convenient to use hue cosmetics such as foundation or pact that is hard to erase when being transferred to the clothes.

### MODE FOR INVENTION

Hereinafter, to facilitate understanding of the present invention, experimental examples are described. However, the following experimental examples are only examples in relation to the effects of the present invention, and the scope and effect of the present invention are not limited thereto.

The following experimental examples used W/O type cosmetic compositions each of which contains six types of thickeners in the internal phase respectively, which are selected in two for each saccharides, anionic thickener, and inorganic thickener.

Specifically, the six types of thickeners are xanthan gum, carbomer, hydroxypropyl starch phosphate, sodium magnesium silicate, hydroxyethyl cellulose, hydroxyethyl acrylate/sodium acryloyldimethyl taurate copolymer.

### Experimental Examples

In the following experimental examples, the transfer improvement effect was confirmed for the six W/0 type cosmetic compositions. Since human skin has individual differences in absorbency, artificial leathers were used in the experimental examples to identify objective transfer. Specifically, a certain amount (0.1 g) of the formulation was applied to the artificial leather using poly-glove, and after 30 minutes, another artificial leather was covered to confirm the degree of transfer. Thereafter, the amount of the transferred make-up was quantified and analyzed by an image analysis program. The results are shown in table 2 below.

TABLE 2

| | thickener added to the internal | Transfer | Improvement |
|---|---|---|---|
| | phase | value | rate (%) |
| External phase film | — | 44234.34 | — |
| former alone | | | |
| External phase film | Xanthan gum | 35942.4 | 18.7 |
| former + Internal | Carbomer | 37677.75 | 14.8 |
| phase thickener | Hydroxypropyl starch phosphate | 40644.03 | 8.1 |
| | Sodium magnesium silicate | 32197.2 | 27.2 |
| | Hydroxyethyl cellulose | 34199.82 | 22.7 |
| | Hydroxyethyl acrylate/sodium | 38039.97 | 14.0 |
| | acryloyldimethyl taurate copolymer | | |

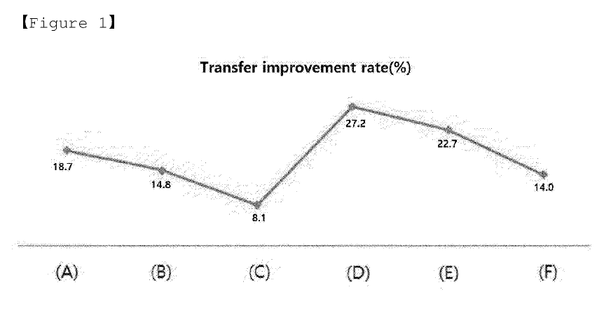

The improvement rates of the result shown in the above table 2 can be more clearly compared through the graph of FIG. 1.

Referring to table 2 and FIG. 1, it can be seen that the formulations containing the thickener in the internal phase have a transfer improvement effect irrespective of the type of the thickener, compared to the formulation to which the film former was exclusively applied in the external phase. The average improvement rate was 17.6%. Especially, it was confirmed that when sodium magnesium silicate and hydroxyethyl cellulose were used as the thickener, improvement rates were 27.2% and 22.7%, respectively, indicating a better transfer improvement effect.

1. A water-in-oil cosmetic composition comprising a film former in an external phase, and a thickener is further contained in an internal phase.

2. The water-in-oil cosmetic composition of claim 1, wherein the thickener contained in the internal phase is at least one thickener selected from the group consisting of saccharides, anionic thickener and inorganic thickener.

3. The water-in-oil cosmetic composition of claim 2, wherein the thickener is at least one selected from the group consisting of xanthan gum, carbomer, hydroxypropyl starch phosphate, sodium magnesium silicate, hydroxyethyl cellulose and hydroxyethyl acrylate/sodium acryloyldimethyl taurate copolymer.

4. The water-in-oil cosmetic composition of claim 2, wherein the thickener is at least one selected from the group consisting of sodium magnesium silicate and hydroxyethyl cellulose.

5. The water-in-oil cosmetic composition of claim 1, wherein the content of the thickener is 0.1˜0.5% by weight based on the total weight of the composition.

6. The water-in-oil cosmetic composition of claim 1, wherein the film former contained in the external phase is trimethylsiloxysilicate or polypropylsilsesquioxane.

7. The water-in-oil cosmetic composition of claim 1, wherein the water-in-oil cosmetic composition has a transfer preventing effect.

8. The water-in-oil cosmetic composition of claim 1, wherein the water-in-oil cosmetic composition is at least one selected from the group consisting of foundation, pact, powder, makeup base, sunscreen, primer and concealer.
